SEQUENCE LISTING (5 sequences)

SEQ ID NO: 1 - protein, 9 aa, Artificial Sequence
  The sequence is synthesized.
PRVDVRVDP

SEQ ID NO: 2 - protein, 9 aa, Artificial Sequence
  The sequence is synthesized.
PKVEVKVEP

SEQ ID NO: 3 - protein, 9 aa, Artificial sequence
  The sequence is synthesized.
PRVDVRVDV

SEQ ID NO: 4 - protein, 13 aa, Artificial sequence
  The sequence is synthesized.
PRVDVRPDVRVDP

SEQ ID NO: 5 - protein, 16 aa, Artificial sequence
  The sequence is synthesized.
RADARADARADARADA

(19) United States
(12) Patent Application Publication
(10) Pub. No.: US 2023/0310702 A1
(43) Pub. Date: Oct. 5, 2023

(54) SELF-ASSEMBLING PEPTIDES, PREPARATION METHODS, SELF-ASSEMBLING PEPTIDE FORMULATIONS AND USE

(71) Applicant: Success Bio-Tech Co., Ltd., Shandong (CN)

(72) Inventors: Shang Zhang, Shandong (CN); Fangmin Li, Shandong (CN); Lichang Gao, Shandong (CN); Pin Wang, Shandong (CN); Jinhui Feng, Shandong (CN); Yuanxue Liu, Shandong (CN); Xudong Zhang, Shandong (CN); Changfeng Liu, Shandong (CN); Chengru Zhao, Shandong (CN); Lili Zhao, Shandong (CN); Junying Guo, Shandong (CN); Zaiqing Zhang, Shandong (CN); Yongli Yan, Shandong (CN); Chunxia Zhang, Shandong (CN); Weiwei Gao, Shandong (CN); Huan Bai, Shandong (CN)

(73) Assignee: Success Bio-Tech Co., Ltd., Shandong (CN)

(21) Appl. No.: 17/638,038

(22) PCT Filed: May 19, 2021
(86) PCT No.: PCT/CN2021/094528

§ 371 Date: Feb. 24, 2022

(30) Foreign Application Priority Data
Aug. 5, 2020 (CN) 202010780592.9

(51) Int. Cl. A61L 27/22; C07K 7/06; A61L 27/54

(52) U.S. Cl. CPC A61L 27/227; A61L 27/54; C07K 7/06; A61L 2300/424; A61L 2400/04; A61L 2430/34

(57) ABSTRACT

Provided is a self-assembling peptide, a preparation method, the self-assembling peptide formulations and use, and it is related to the field of biotechnology. The self-assembling peptide provided by the present disclosure has a general formula as shown in AC-Pro-X1-X3-X2-X3-X1-X3-X2-Pro-amide. Through experiments, the inventors of the present disclosure found that the self-assembling peptide provided by the present disclosure, because solubility is high and difficulty of synthesis and purification is less compared with a traditional self-assembling peptide, is more feasible for industrial production, and even cheaper to produce. In mass synthesis and purification, the number of purification decreases, the single purification amount increases, the purity of a crude peptide can reach 90% or more, and the cost is greatly reduced.

### TECHNICAL FIELD

The present disclosure relates to the field of biotechnology, and particularly relates to self-assembling peptides, the preparation methods, the self-assembling peptide formulations and use.

### SEQUENCE LISTING

The instant application contains a Sequence Listing which has been submitted electronically in ASCII format and is hereby incorporated by reference in its entirety. Said ASCII copy, created on Oct. 17, 2022 is named P03807 _SL_ST25.txt and is 1.41 kilobytes in size.

### BACKGROUD

Self-assembly techniques are novel bio-nanotechnologies, among which the self-assembling peptide area is one research focus. And multiple types of self-assembling peptides successively showed unique properties thereof. An ionic complementary self-assembling peptide has a structure of alternating hydrophilic and hydrophobic amino acids, presents in a β-sheet conformation in a water solution, and has a hydrophilic surface formed by charged amino acids and a hydrophobic surface formed by hydrophobic amino acids. Such a special conformation makes peptide molecules self-assemble and form nanofibers, and after reaching a triggering condition such as contacting with metallic ions, assembly will be continuing to form a hydrogel. The water content of the hydrogel formed by such a peptide self-assembling can reach 99%, and the hydrogel also has good biocompatibility.

The self-assembling peptides are used in a variety of applications, including three-dimensional cell culture scaffolds, hemostatics, hemostatics and anti-adhesion agents for sinus surgery, mucosal fillers, 3D printing and tissue engineering scaffolds, controlled drug release, etc. The self-assembling peptides have been developed as medical products such as self-assembling peptide hemostatics and hydrogel dressings, etc., and a commercialized self-assembling peptide sequence is AC-RADARADARADARADA-amide abbreviated as AC-(RADA)₄-amide or RADA16 (CN101267831, CN106459154A), and in addition, the commonly-used self-assembling peptide sequences also includes IEIK13 (CN106459154A), KLD12 (CN106459154A), etc. These self-assembling peptides are mostly composed of alternately arranged positively charged amino acid, negatively charged amino acid and hydrophobic amino acid. In which one typical application is using the self-assembling peptide as a gastrointestinal mucosa hemostatic, that is to say, delivering a self-assembling peptide solution to the stomach or intestinal tract through a long catheter and releasing the self-assembling peptide solution to a wound surface; when this self-assembling peptide solution is in contact with a wound surface of the gastrointestinal tract, because blood in the wound surface contains metal ions which trigger the self-assembling peptide to assemble into a glue, a solution state during the delivery is changed into a gel state, resulting in its physical hemostasis effect.

The application of the self-assembling peptide is influenced by its own properties. For instance, during application procedures, RADA16 is prepared into an aqueous solution with a high concentration such as 1% or 2%, then the solution turns to a viscous liquid within a very short time, although the viscous liquid also retains a certain fluidity, the viscous liquid is still not easy to deliver in a long catheter, which limits its application as a self-assembling material. Ideally, such materials need to be completely in a solution state before self-assembling into a gel, that is to say, the viscosity is low or completely close to the viscosity of water, and then the material forms a gel after contact with a triggering agent or reaching a triggering condition during application procedures; as a result, this can ensure that the self-assembling peptide is easy to deliver in a pipeline or a passage during delivery, and can ensure that at the point the self-assembling peptide as a hemostatic is in contact with a wound surface to stop the bleeding, it is easy for the self-assembling peptide to spread on the wound surface and conform to the wound shape in the process that the self-assembling peptides is in contact with blood to trigger gelation,. Accordingly, such self-assembling still needs a further study to promote its application.

In view of this, the present disclosure is proposed hereby.

### SUMMARY

A main purpose of the present disclosure is to provide a type of self-assembling peptides, to solve at least one of the technical problems in the prior art.

In order to achieve the above purpose, the present disclosure provides a type of self-assembling peptides, wherein the self-assembling peptide has a general formula of:AC-Pro-X1-X3-X2-X3-X1-X3-X2-Pro-amide;
where, the N terminal is acetyl, and the C terminal is an amide group;
X1 is independently a positively charged amino acid, X2 is independently a negatively charged amino acid, and X3 is independently a hydrophobic amino acid.

Further, the X1s comprise one or more of Lys, Arg or His.

Further, the X2s comprise Asp and/or Glu.

Further, the X3s comprise one of Val, Leu, Ile or Phe.

The present disclosure also provides a preparation method for the above-mentioned self-assembling peptide, wherein the preparation method includes a solid-phase peptide synthesis method.

The present disclosure also provides self-assembling peptide formulations, which contain the above-mentioned self-assembling peptides.

Further, the formulations of the self-assembling peptides include a powder form or a solution form.

Further, the self-assembling peptide formulations described herein also include the pharmaceutically acceptable carriers and/or excipients.

In addition, the present disclosure also provides applications of the above-mentioned self-assembling peptides or the self-assembling peptide formulations in any one of (a)-(c):
(a) preparing a hemostatic material;
(b) preparing a mucosal filler; and
(c) preparing an anti-adhesion agent.

Compared with the prior art, the present disclosure has the following beneficial effects:

the self-assembling peptide provided by the present disclosure has the general formula as shown in AC-Pro-X1-X3-X2-X3-X1-X3-X2-Pro-amide; by designing a head end and a tail end of the self-assembling peptide as Pro, since the formation of a random coil can be promoted by the pyrrole ring on a Pro side chain, it increases the solubility of the peptide and benefits the condensation of amino acids during peptide synthesis. Meanwhile, the self-assembling peptide sequence contains alternately arranged positively charged amino acid, negatively charged amino acid and hydrophobic amino acid within a same sequence, so that a stable aggregate can be spontaneously formed between the peptide molecules by a non-covalent bonds such as a hydrogen bond, an electrostatic interaction, or a hydrophobic interaction, subsequently a gel forms at the point of contacting with metallic ions or when the changing of the pH value of the solution or the changing of the osmotic pressure of the solution.

Through experiments, the inventors of the present disclosure found that, for the self-assembling peptide provided by the present disclosure, because its solubility was better and the difficulty of synthesis and purification was less compared with a classic self-assembling peptide, it was more feasible for industrial production, and even cheaper to produce. As a result, in massive synthesis and purification, the number of purification procedures decreased, the yield of a single purification procedure increased, the purity of a crude peptide could reach 90% or more, and the cost was greatly reduced.

### BRIEF DESCRIPTION OF THE DRAWINGS

In order to more clearly explain the specific embodiments of the present disclosure or the technical solution in the prior art, the accompanying drawings required for description of the specific embodiments or the prior art is introduced briefly below. Evidently, the accompanying drawings in the following description are some embodiments of the present disclosure, for those of ordinary skill in the art, other accompanying drawings can be obtained on the basis of these accompanying drawings without performing a creative work.

FIG. 1 is the high performance liquid chromatography graph of the crude peptide having a peptide sequence ① provided by Example 1 of the present disclosure;

FIG. 2 is the high performance liquid chromatography graph of a synthesized peptide sequence ① after purification provided by Example 1 of the present disclosure;

FIG. 3 is the mass spectrogram of a peptide sequence ① provided by Example 1 of the present disclosure;

FIG. 4A shows the storage modulus of a peptide sequence ① provided by the present disclosure under a condition of frequency scanning at 0.1 hz-10 hz;

FIG. 4B shows the storage modulus of a peptide sequence ② provided by the present disclosure under a condition of frequency scanning at 0.1 hz-10 hz;

FIG. 4C shows the storage modulus of a peptide sequence ③ provided by the present disclosure under a condition of frequency scanning at 0.1 hz-10 hz;

FIG. 4D shows the storage modulus of a peptide sequence ④ provided by the present disclosure under a condition of frequency scanning at 0.1 hz-10 hz;

FIG. 4E shows the storage modulus of peptide sequence ⑤ provided by the present disclosure under a condition of frequency scanning at 0.1 hz-10 hz;

FIG. 5A shows the storage modulus of a peptide sequence ① provided by the present disclosure under a condition of frequency scanning at 1 hz;

FIG. 5B shows the storage modulus of a peptide sequence ② provided by the present disclosure under a condition of frequency scanning at 1 hz;

FIG. 5C shows the storage modulus of a peptide sequence ③ provided by the present disclosure under a condition of frequency scanning at 1 hz;

FIG. 5D shows the storage modulus of a peptide sequence ④ provided by the present disclosure under a condition of frequency scanning at 1 hz;

FIG. 5E shows the storage modulus of a peptide sequence ⑤ under a condition of frequency scanning at 1hz;

FIG. 6A shows a rabbit back skin wound bleeding condition about 20 s after spraying a wound with a peptide aqueous solution according to Example 4 of the present disclosure;

FIG. 6B shows a rabbit back skin wound bleeding condition after removing a hydrogel covering the surface according to Example 4 of the present disclosure;

FIG. 7A shows a liver wound bleeding condition about 15 s after spraying the wound with a peptide aqueous solution according to Example 5 of the present disclosure;

FIG. 7B shows a liver wound bleeding condition after removing an excess hydrogel according to Example 5 of the present disclosure;

FIG. 8A is a graph showing a hematoxylin and eosin staining result of mucosae of posterior gastric walls of rabbits injected with normal saline according to Example 6 of the present disclosure;

FIG. 8B is a graph showing a hematoxylin and eosin staining result of mucosae of posterior gastric walls of rabbits injected with 0.5 ml of a 10 mg/ml sodium hyaluronate solution according to Example 6 of the present disclosure;

FIG. 8C is a graph showing a hematoxylin and eosin staining result of mucosae of posterior gastric walls of rabbits injected with 0.5 ml of a 3% peptide aqueous solution ① according to Example 6 of the present disclosure;

FIG. 9A is a graph showing a hematoxylin and eosin staining result of an untreated rabbit colonic mucosa according to Example 7 of the present disclosure;

FIG. 9B is a graph showing a hematoxylin and eosin staining result of colonic mucosa of rabbits injected with normal saline according to Example 7 of the present disclosure;

FIG. 9C is a graph showing a hematoxylin and eosin staining result of colonic mucosa of rabbits injected with 0.5 ml of a 10 mg/ml sodium hyaluronate solution according to Example 7 of the present disclosure; and

FIG. 9D is a graph showing a hematoxylin and eosin staining result of colonic mucosa of rabbits injected with 0.5 ml of a 3% peptide aqueous solution ① according to Example 7 of the present disclosure.

### DETAILED DESCRIPTION

A technical solution of the present disclosure will be clearly and completely described below in conjunction with examples. Obviously, the described examples are a portion of examples of the present disclosure, and not all of the examples. All other examples obtained by those of ordinary skill in the art based on the examples of the present disclosure without paying a creative work all belong to the protection scope of the present disclosure.

Unless otherwise defined herein, the scientific and technical terms used along with the present disclosure should have a meaning commonly understood by those of ordinary skill in the art. The meaning and range of the terms should be clear, however, in any case of potential ambiguity, a definition provided herein has a priority over any dictionaries or foreign definitions. In the present application, unless otherwise stated, use of the term “include” and other forms is nonrestrictive.

Generally, the nomenclature and its technology used along with cell and tissue culture, molecular biology, immunology, microbiology, genetics as well as protein and nucleic acid chemistry and hybridization described herein are those well known and commonly used in the art. Unless otherwise stated, the method and technology of the present disclosure are generally executed according to the conventional method well known in the art and descried in a general and more specific reference, the references are quoted and discussed throughout this description. An enzymatic reaction and a purification technology are executed according to an instruction of a manufacturer, as normally implemented in the art or as descried herein. The nomenclature used along with analytical chemistry, synthetic organic chemistry as well as medicine and pharmaceutical chemistry described herein, as well as a laboratory procedure and technology thereof are those well known and commonly used in the art.

Current self-assembling peptides are mainly represented by AC-(RADA)₄-amide. After an extensive research and screening, the present disclosure developed a series of novel self-assembling peptide sequence, one of major characteristics thereof is in that, two ends of the self-assembling peptide sequence, i.e. an amino terminal and a carboxyl terminal contain proline. The proline is referred to as Pro or P.

Specifically, according to a first aspect of the present disclosure, herein provided a type of self-assembling peptides, wherein the self-assembling peptide has a general formula of:
AC-Pro-X1-X3-X2-X3-X1-X3-X2-Pro-amide;
where, the N terminal is acetyl, and the C terminal is an amide group;
X1 is independently a positively charged amino acid, X2 is independently negatively charged amino acid, and X3 is independently a hydrophobic amino acid.

For the self-assembling peptide of the present disclosure, the head and tail are particularly designed as Pro, a pyrrole ring at a Pro side chain contributes to the formation of a random coil, which can increase solubility of the peptide, and benefits the condensation of the amino acids when a peptide is synthesized. In the synthesis process of the existing AC-(RADA)₄-amide self-assembling peptide, repeated feeding is usually required, hence the yield reduces, also resulting in complex by-products, such a crude peptide is generally to be dissolved to 0.1% or even lower concentration for purification thus the purification difficulty is huge, and the cost is high. For the peptide having the amino acid Pro provided by the present disclosure, only single feeding is required for a complete reaction at each site, the difficulty of synthesis is lower, the yield is high, the purity of the crude peptide can reach 90%, and the solubility of the crude peptide is high, which benefits the industrial production. By the comprehensive comparisons of the storage modulus and the viscosity of the peptides, the best combination was found when two Pro were added separately at the head and the tail positions in one peptide. Meanwhile, the self-assembling peptide sequence contains alternately arranged positively charged amino acid, negatively charged amino acid and hydrophobic amino acid within the same sequence, so that a stable aggregate can be spontaneously formed between the peptide molecules by non-covalent bonds such as a hydrogen bond, an electrostatic interaction, hydrophobic interaction, etc., subsequently a gel forms at the point of contacting with metallic ions, or when the changing of the pH value of the solution or the changing of the osmotic pressure of the solution.

Through experiments, the inventor of the present disclosure found that, for the self-assembling peptide provided by the present disclosure, because its solubility was better and the difficulty of synthesis and purification was less compared with a classic self-assembling peptide, it was more feasible for industrial production, and even cheaper to produce. As a result, in mass synthesis and purification, the number of purification procedures decreased, the single purification yield increased, the purity of a crude peptide could reach 90% or more, and the cost was greatly reduced.

In some preferred embodiments, the X1s comprise one or more of Lys, Arg or His, for example, the X1s can concurrently be Lys, Arg, or His, or among which one is Lys and the other is Arg, or among which one is Lys, the other is His, etc., and the present disclosure has no limit on this.

In some preferred embodiments, the X2s comprise Asp and/or Glu, for example, the X2s can concurrently be Asp or Glu, or among which one is Asp and the other is Glu, and the present disclosure has no limit on this.

In some preferred embodiment, the X3s comprise one or more of Val, Leu, Ile or Phe, for example, the X3s can concurrently be Val, Leu, Ile or Phe, or can respectively be Val, Leu, and Ile, or Leu, Ile, and Phe, or among which two are Val and the other is Leu, or among which two are Ile and the other is Phe, etc., and the present disclosure has no limit on this.

According to a second aspect of the present disclosure, there is provided a preparation method for the above-mentioned self-assembling peptide, wherein the preparation method includes a solid-phase peptide synthesis method.

The self-assembling peptide of the present disclosure has a simple synthetic process and low cost, which is more beneficial for industrial production, and meanwhile also provides a nanomedical material for more options.

In one specific embodiment, preparation can be carried out in the following manner:

Fmoc protected amino acids are used as raw materials, Rink Amide-MBHA Resin is chosen as the resin, a protective group on the resin is cleaved with 20% piperidine/DMF to link a first amino acid, a condensation agent is TBTU and HOBT, and whether the reaction is complete is checked by a Kaiser reagent. Each amino acid is successively linked in an order of from a C terminal to a N terminal, after a protective group of the last amino acid is cleaved, the N terminal is acetylated with acetic anhydride, and DIEA. After the synthesis is finished, the product is washed alternately with methanol, and dichloromethane for 5 times, and suction filtered under a reduced pressure overnight to remove the organic solvent. Peptide cleaving reagent ratio: TFA:water:TIS = 95:2.5:2.5, a cleaving reagent is added dropwise into precooled absolute diethyl ether, and filtering is conducted by a G4 funnel to obtain a crude peptide. The crude peptide is isolated and purified by preparative HPLC, and freeze-dried to obtain a refined peptide product with a purity greater than 95%.

According to a third aspect of the present disclosure, self-assembling peptide formulations are provided, wherein the self-assembling peptide formulations comprise the above-mentioned self-assembling peptides.

The self-assembling peptide of the present disclosure can be prepared into various formulations for use, for example, the self-assembling peptide can be made as a powder or a liquid formulation for use alone, and can also be mixed with chitin, collagen, starch, etc. to be applied as a spray, a paste or a hydrogel. In addition, this self-assembling peptide can also be present as a coating of a device, such as a scaffold, a catheter, etc. The self-assembling peptide can be dispersed or absorbed in a bandage or foam, so as to play a role in hemostasis and infection prevention. The self-assembling peptide is used in combination with a vasoconstrictor, colorant, analgesic or anesthetic, etc., which can be mixed together to make a preparation, and can also be individually wrapped.

It should be noted that, for the self-assembling peptide formulations provided by the present disclosure, the self-assembling peptide can be adjusted into any concentrations according to an actual need, e.g. 0.1%-99%, the present disclosure has no limit on this. In some preferred embodiments, the self-assembling peptide is present at a concentration of no greater than 4%, for example the concentration can be, but is not limited to, 4%, 3%, 2%, 1% or other numerical values distributed among the above-mentioned numerical values.

Based on that the self-assembling peptide of the present disclosure has a high solubility, when the self-assembling peptide was prepared at a concentration of 1%-4%, the self-assembling peptide still showed a good fluidity, whereas a commercialized AC-(RADA)₄-amide peptide at a concentration of 2.5% had almost lost fluidity. For the comparison of its rheological property, after contact with a metallic ion or change pH value so as to trigger the self-assembly, the storage modulus of the peptide having Pro amino acids could reach 1000 pa or more, whereas the storage modulus of AC-(RADA)₄-amide at a concentration of 2.5% was still less than 700 pa. If necessary, the self-assembling peptide of the present disclosure requires the increasing of the solution concentration to reach a high intensity, but the synthesis and purification are simple and practicable, whereas the final cost does not increase. The AC-(RADA)₄-amide peptide still showed a high viscosity even under a concentration as low as 1%, but the peptide concentration in a clinical application is up to 2.5%, as a result, in an endoscopy procedure, it is laborious to deliver a liquid with a high viscosity using a catheter, and the administration dose is thus not easy to control; a peptide with Pro has a lower viscosity but can reach the same storage modulus, hence the peptide is more convenient to use.

In addition, the self-assembling peptide provided by the present disclosure, under a condition of contacting with metallic ions, self-assembles to form a hydrogel with a nanofiber network structure, and has a good water retention and air permeability, which can achieve an aim of rapid hemostasis, and can provide an advantageous environment for wound healing. When a solution of the self-assembling peptide provided by the present disclosure is applied onto a bleeding site or skin wound surface, it can rapidly form a gel after contact with the body fluid containing metallic ions, and the bleeding site or skin wound surface is rapidly sealed, so as to play a role in hemostasis and wound care. The synthetic peptides have no immunogenicity, and their metabolites are natural amino acids, thus the synthetic peptides can be absorbed and utilized by a human body.

Based on this, a fourth aspect of the present disclosure provides the use of the above-mentioned self-assembling peptide or the above-mentioned self-assembling peptide formulations.

Optionally, the self-assembling peptide provided by the present disclosure can be used as hemostatic materials: designed and synthesized peptides are different from raw materials obtained from natural materials for the peptide is safer excluding the potential of immunogenicity; efficient and rapid hemostatic effects can be achieved, and in an animal experiments, the hemostasis of skin and liver were all achieved within more than ten seconds; various dosage forms such as solid or liquid preparations can be used in a catheter and a spray bottle, being convenient and efficient; a liquid preparation is able to conform to and fill an irregular wound surface, without a need for any tissue compression, can be used in hemostasis of organs in the body and brain, and can also be used in hemostasis of a body surface. When the self-assembling peptide is used on an acute or chronic wound surface, not only bleeding can be rapidly stopped, with a water content up to 99%, but also it can keep the wound wet and suits a dry wound surface. Different from a traditional hydrogel, a peptide hydrogel has a combined structure of nanofiber mesh and water, absorbs exudates and segregates contaminants, and has a certain gas permeability, thereby promoting wound healing.

Optionally, the self-assembling peptide provided by the present disclosure can be used as a mucosal filler. An endoscopic mucosal resection is a commonly-used minimally invasive technique, due to its simplicity and safety, and can remove large polyp (greater than 22 cm) and early tumor, thus the endoscopic mucosal resection is widely used. Usually, by submucosal injection, a cushion layer is established between a surface mucosa and a muscular tissue layer to elevate the mucosa for assisted resection. Since 1984, normal saline (0.9 wt% sodium chloride) has always been a main injection for endoscopic mucosal resection. Recently, other fluids including hypertonic saline, hypertonic glucose aqueous solution, autoblood, sodium hyaluronate, glycerol, hyaluronic acid, succinylated gelatin, hydroxypropyl methyl cellulose, poloxamer and fibrinogen have been used to prolong the stability of the cushion layer by increasing the viscosity of a fluid. But the application of these solutions is largely limited by unsatisfied safety and duration. Specifically, hypertonic saline, dextrose aqueous solution and glycerol results in a decreased height of the cushion layer of less than 50% after 30 minutes. For example, a carboxymethyl cellulose solution, due to its high viscosity, may require a special 18 gauge submucosal injection needle catheter, to decrease injection resistance to the maximum extent. Furthermore, hyaluronic acid potentially stimulates growth of the residual tumor tissue. Fibrinogen and autoblood are biomaterials, and can potentially increase an infection risk caused by the contaminations. The submucosal injections play a crucial role in a successful, safe and complete removal of a lesion, because the submucosal injections can not only elevate a diseased mucosa but also provide a certain void between the surface mucosa and the muscular tissue layer, which contributes to the removal of lesions. Ensuring complete and safe resection can reduce the risk of local recurrence, hence, an ideal injection for submucosal elevation must be biocompatible, easier for injection and able to provide a durable submucosal cushion. Compared with an existing technology, the mucosal lifting agent of sodium hyaluronate used in a endoscopy of the digestive duct, the self-assembling peptide provided by the present disclosure or its hydrogel formulations are easier to operate, the fluidity of its aqueous solution is better, and it is easier to deliver and to inject. The self-assembly does not occur until the contact with a body fluid, avoiding a risk of the jamming of the injection needle. This peptide aqueous solution will form gel in contacting with the body fluid, without the need of any additional interventions such as lighting or shearing forces. In addition, the self-assembling peptide hydrogel formulation has a certain intensity, and a strong water retention function, which makes the gel maintain a certain thickness lasting for a long time, avoiding repeated injections. As a result, after the dissection of the diseased mucosa, the self-assembling peptide hydrogel formulation will still maintain a solid shape and will not flow outwards.

Optionally, the self-assembling peptide provided by the present disclosure can be used as an anti-adhesion agent. Due to the high water content of the self-assembling peptide of the present disclosure, the self-assembling peptide is able to occupy a certain space, and has a certain of lubrication and anti-adhesion functions.

The present disclosure will be further explained by specific examples, but it should be understood that these examples are merely used to explain in more detail, and should not be understood to limit the present disclosure in any way.

It should be noted that, in the examples of the present disclosure, “AC-” in the peptide sequence indicates that it is acetyl, and “-amide” indicates that it is an amide group, which are not thus embodied in the sequence list.

### Example 1. Synthesis of Peptides

### Synthesis of a Peptide of a Sequence ① (SEQ ID NO.1):AC-Pro-Arg-Val-Asp-Val-Arg-Val-Asp-Pro-amide (or abbreviated as AC-PRVDVRVDP-amide)

### 1. Materials

Fmoc-Pro-OH (N-fluorenylmethoxycarbonyl-proline), Fmoc-Val-OH (N-fluorenylmethoxycarbonyl-valine), Fmoc-Arg(pbf)-OH (N-fluorenylmethoxycarbonyl-2,2,4,6,7-pentamethyldihydrobenzofuran-5-sulfonyl-arginine, Fmoc-Asp(OtBu)-OH (N-fluorenylmethoxycarbonyl-4-tert-butyl ester-aspartic acid), Rink Amide-MBHA Resin (resin), TBTU (O-benzotriazol-1-yl-N,N,N′,N′-tetramethyluronium tetrafluoroborate), and HOBT (1-hydroxylbenzotriazole) were purchased from GL Biochem (Shanghai) Co., Ltd. Piperidine, acetic anhydride, DMF (N,N-dimethylformamide), TFA (trifluoroacetic acid), NMM (N-methylmorpholine), diethyl ether, methanol and DCM (dichloromethane) were purchased from Sinopharm Chemical Reagent (Beijin) Co., Ltd.

### 2. Synthesis Method

Using a Fmoc solid-phase synthesis method: 1 g of Rink Amide-MBHA Resin was weighed and soaked in 24 ml DCM overnight, and suction filtrated under a reduced pressure to remove DCM, DMF was added to wash the filtrated material twice, 30 ml of a 20% piperidine/DMF solution was added, after 30 min, suction filtration was performed under a reduced pressure, and the filtrated material was washed with DMF and methanol successively. Whether the resin protective group was removed thoroughly was checked using ninhydrin. 540 mg of Fmoc-Pro-OH, 432.4 mg of HOBt, 488.4 mg of TBTU, 352 µl of NMM were added, 30ml of DMF was added, and a reaction was carried out for 2 hours, a small number of resin was taken to check whether the Pro was fully linked, after the Pro was fully linked, the 20% piperidine/DMF solution was added, the Fmoc protective group of amino acid was removed, for each amino acid, the above steps were repeated from the C terminal to the N terminal. After all amino acids were linked, the protective group of the last amino acid was cut off. 2ml of acetic anhydride and 3.2 ml of DIEA were added, the reaction was carried out for 20 min, and the N terminal was acetylated. After repeated washing with DCM and DMF, vacuum drying was performed overnight. 20ml of TFA cleaving fluid was added, the cleaving fluid was added dropwise into pre-cooled absolute ethyl ether for extraction, a white flocculent precipitate was produced, and filtered with a G4 funnel, and a filter cake was freeze-dried to obtain crude peptide powder. The crude peptide powder was purified by high performance liquid chromatograph, target peaks were collected, and freeze-drying was conducted to obtain a refined product. It can be seen from a HPLC chromatogram of the crude peptide; the content of the refined peptide may reach 90%. Mass spectrometry shows that the synthesized peptide has a conformed characterization.

Peptides with multiple combinations of sequences can be synthesized according to this method:
AC-Pro-Lys-Val-Glu-Val-Lys-Val-Glu-Pro-amide sequence ② (SEQ ID NO.2);
AC-Pro-Arg-Val-Asp-Val-Arg-Val-Asp-Val-amide sequence ③ (SEQ ID NO.3);
AC-Pro-Arg-Val-Asp-Val-Arg-Pro-Asp-Val-Arg-Val-Asp-Pro-amide sequence④ (SEQ ID NO.4).

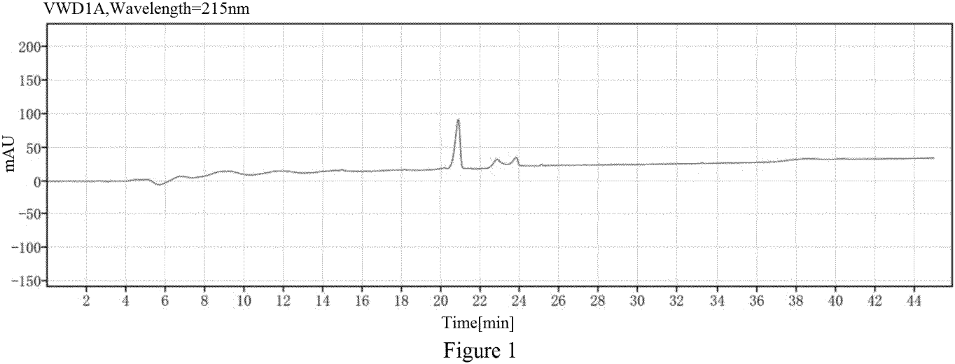

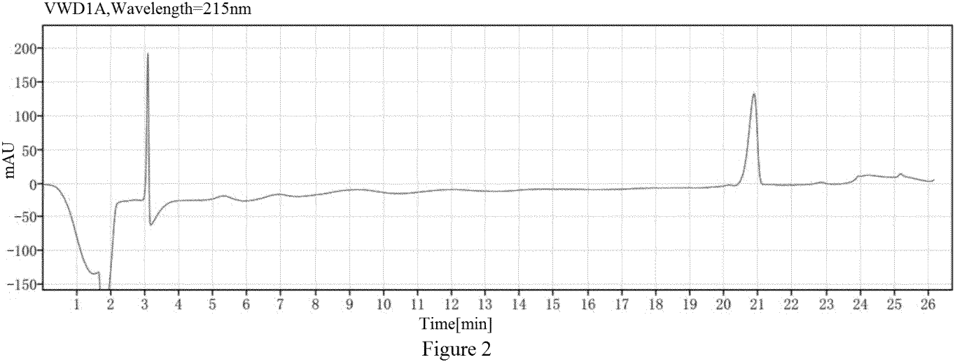

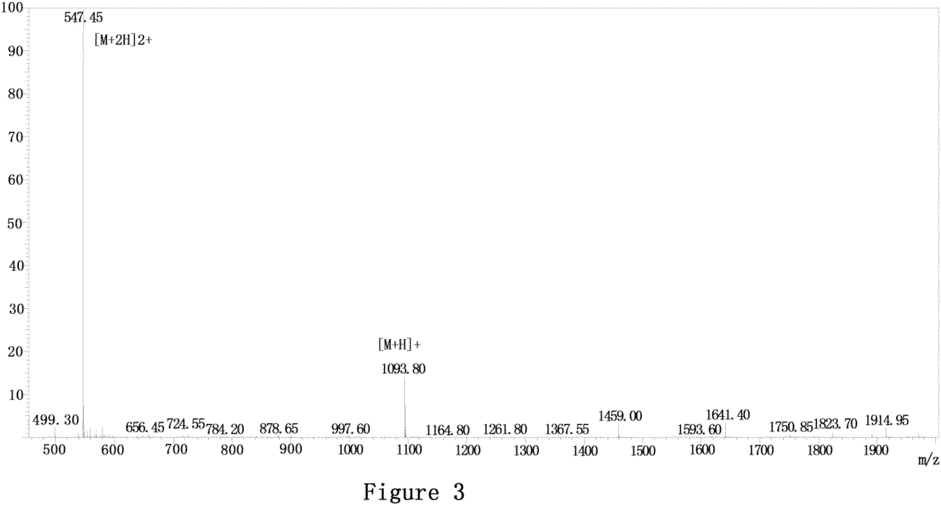

HPLC chromatographic analysis was performed for the crude peptide, chromatography and mass spectrometry test were performed for the refined peptide product, the experiment results are as shown in FIG. 1 to FIG. 3, where FIG. 1 is the high performance liquid chromatography graph of the crude peptide having a peptide sequence ①, it can be seen from the drawing that the impurity content in the crude peptide remains less, and the purity reaches 95%. FIG. 2 is the high performance liquid chromatography graph of a synthesized peptide sequence ① after purification; after purification, no obvious impurity was detected. FIG. 3 is a mass spectrogram of a peptide sequence ①; its molecular weight is 1092.8 Da, conforming to a theoretical molecular weight of this sequence.

### Example 2. Rheological Property Detection

Peptide solutions with different concentrations were prepared, and an appropriate amount of 10×PBS was added such that the final PBS concentration of the solution was 1×, after the solution was in contact with PBS to be triggered to be gel, the gel was taken out slowly with a spoon and placed onto a rheometer plate. A 40 mm plate was placed leaving the gap of about 450 µm, and kept at 37° C. for 5 minutes under a pressure of 1 pa, and frequency scanning was at 0.1 hz-10 hz. The viscosity of the peptides ③, ④ and ⑤ (SEQ ID NO.5) increased with the increase of the concentrations during dissolution. The highest concentration of the solution was set with the reference to the highest concentration at which a gel will not form immediately when the peptide was fully dissolved, according to which peptide solutions with a serial of diluted concentrations were prepared to perform storage modulus detections. For peptides represented by ① and ② in which the head and the tail are Pro, the storage modulus were able to reach 1000pa or higher after the PBS triggered the gelation, whereas before gelation, the solution can also maintain good fluidity.

Data of solution concentrations:

| Sequence information | Preparation concentration |
|---|---|
| ① AC-Pro-Arg-Val-Asp-Val-Arg-Val-Asp-Pro-amide | 1%, 2%, 3%, 4% |
| ② AC-Pro-Lys-Val-Glu-Val-Lys-Val-Glu-Pro-amide | 1%, 2%, 3%, 4% |
| ③ AC-Pro-Arg-Val-Asp-Val-Arg-Val-Asp-Val-amide | 1%, 2% |
| ④ AC-Pro-Arg-Val-Asp-Val-Arg-Pro-Asp-Val-Arg-Val-Asp-Pro-amide | 1%, 2% |
| ⑤ AC-Arg-Ala-Asp-Ala-Arg-Ala-Asp-Ala-Arg-Ala-Asp-Ala-Arg-Ala-Asp-Ala- amide | 1%, 2%, 2.5% |

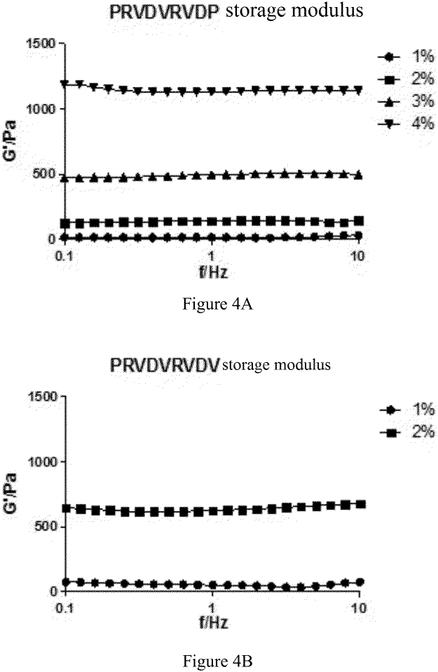

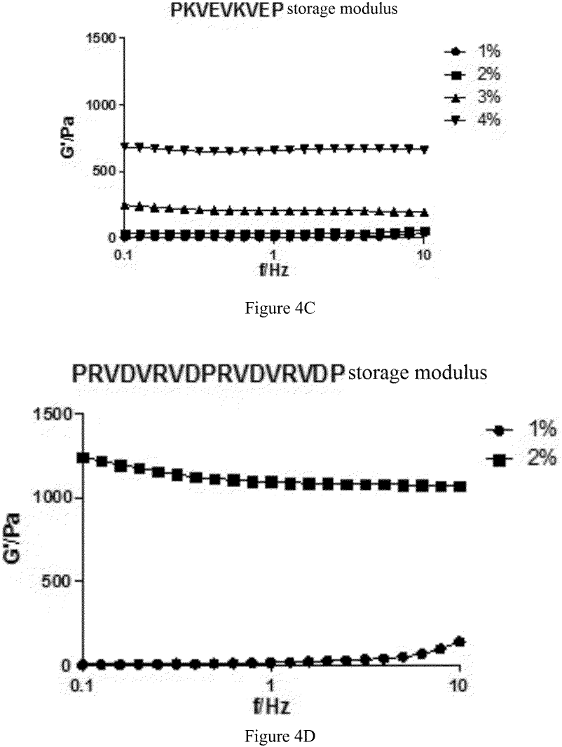

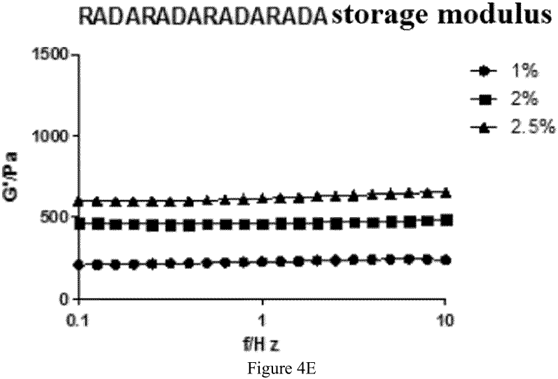

The results of the storage modulus under frequency scanning at 0.1 hz-10 hz are as shown in FIG. 4A-FIG. 4E. It can be seen from the drawings that under the highest concentration the storage modulus of the peptides in which two ends are P can be up to 1000 Pa or higher, which are higher than the storage modulus of a commonly-used sequence of AC-(RADA)₄-amide in which two ends have no P under their highest concentration, indicating that the sequence within which two ends are P can reach a highest intensity at its highest concentration, having a greater application potential.

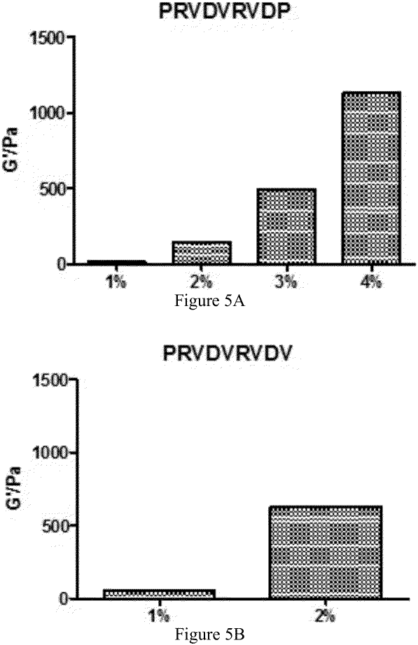

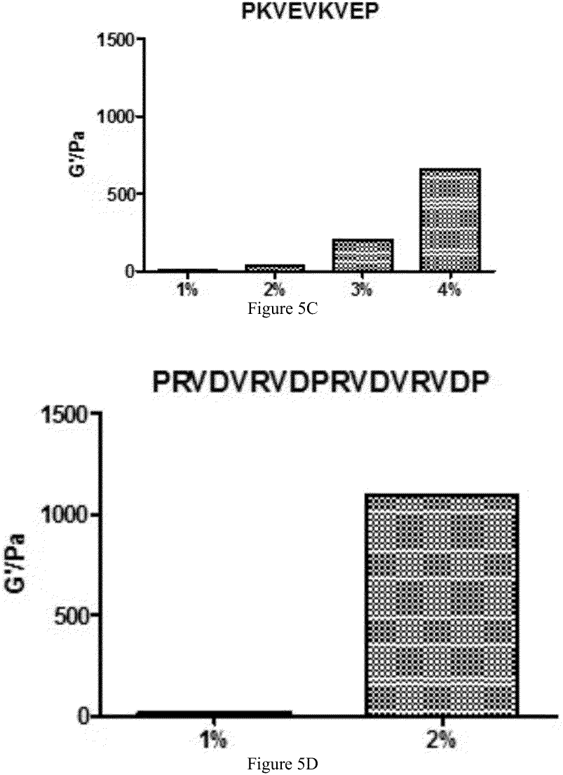

The results of the storage modulus under frequency scanning at 1hz are as shown in FIG. 5A-FIG. 5E, consistent with the results shown in FIG. 4A-FIG. 4E, it can be intuitively seen that, the sequence in which two ends are P has an ability of forming a gel with a higher intensity.

### Example 3. Viscosity Testing

A peptide solution was prepared, a 50 mm plate was chosen for the rheometer, a 1 mm gap was set, and 2 ml of the peptide solution was taken, and placed in the gap under the plate. The shear rate was set as 10 1/s. Average values were recorded. No viscosity numerical value was detected in ① and ② under a concentration of 4%, reflecting a good fluidity of the solution. Whereas, AC-RVDVRVDV-amide and AC-(RADA)₄-amide have high viscosities under a concentration of 1%.

| Sequence | Concentration | Viscosity (mPa·s) |
|---|---|---|
| ① AC-Pro-Arg-Val-Asp-Val-Arg-Val-Asp-Pro-amide | 3% | Under the detection limit |
| | 4% | Under the detection limit |
| ② AC-Pro-Lys-Val-Glu-Val-Lys-Val-Glu-Pro-amide | 3% | Under the detection limit |
| | 4% | Under the detection limit |
| AC-Arg-Val-Asp-Val-Arg-Val-Asp-Val-amide | 1% | 658.25 |
| | 2% | 1546.21 |
| ⑤ AC-Arg-Ala-Asp-Ala-Arg-Ala-Asp-Ala-Arg-Ala-Asp-Ala-Arg-Ala-Asp-Ala-amide | 1% | 955.17 |
| | 2% | 1817.96 |
| | 2.5% | 2514.57 |

### Example 4. Rabbit Hemostasis Experiment on the Back

Hemostatic procedures: a peptide aqueous solution ① with a concentration of 3% was prepared for rabbit hemostasis on the back. After injection of anesthesia via ear veins in New Zealand rabbits, a skin incision of about 1.5 cm was made on the back, and an incision depth was made until a blood vessel ruptured, and blood flowed out. After the blood flowed out was wiped off with a gauze, the peptide aqueous solution was sprayed onto a wound site immediately, and timing was conducted. In the hemostatic process, oozed blood was wiped constantly with a gauze, until no blood flowed out, and the timing was stopped, and after 1 minute, excess gel was removed, and the wound was observed whether the blood further oozed out.

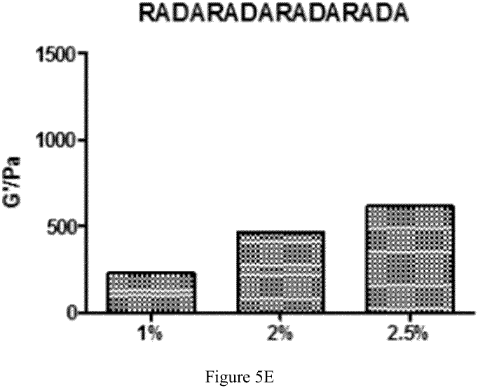

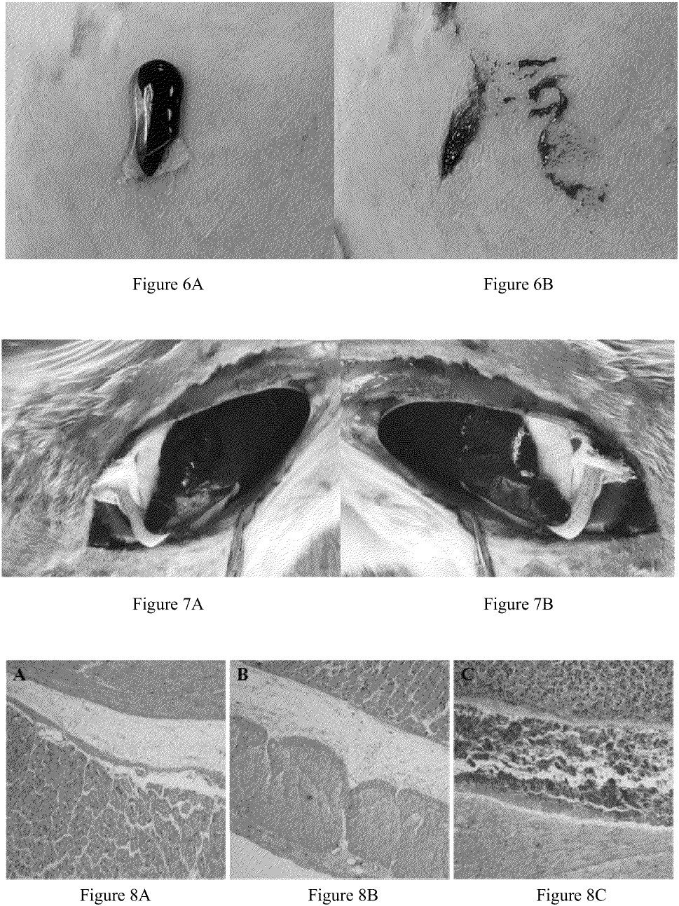

Results: after the peptide aqueous solution was sprayed onto the wound, a gel was formed immediately, after about 20 s, no blood flowed out (FIG. 6A), after the hydrogel covering the surface was removed, the wound was clearly visible, and no blood oozed out (FIG. 6B).

### Example 5. Rabbit Liver Hemostasis Experiment

Hemostatic process: a peptide aqueous solution ① with a concentration of 3% was prepared, abdomen was opened after anesthesia of the rabbit, liver was exposed, and after the hepatic vessel was lacerated, the blood flowed out. After the blood flowed out was wiped off with a gauze, the peptide aqueous solution was sprayed onto the wound site immediately, and timing was conducted. In the hemostatic process, the oozed blood was constantly wiped with a gauze, until no blood flowed out, and the timing was stopped, and after 1 minute excess gel was removed, and the wound was observed to examine whether the blood further oozed out.

Results: after the peptide aqueous solution was sprayed onto the wound, when the peptide aqueous solution was in contact with blood, a gel was formed immediately, after about 15 s, bleeding was completely stopped (FIG. 7A), and after the excess hydrogel was removed, hemostasis still obtained (FIG. 7B).

### Example 6. Rabbit Stomach Submucosal Injection

After a New Zealand rabbit was fastened for 24 hours, injection anesthesia was conducted from the ear vein, the abdomen was then opened, afterwards a stoma was made in the anterior wall of the stomach to expose a stomach mucosa, and 0.5 ml of normal saline was injected into a posterior submucosal layer using a 25G needle. After being observed for 30 min, the rabbit was sacrificed, a tissue was then taken out and fixed in formalin for 2 days, embedded by paraffin, sectioned, and finally stained with hematoxylin and eosin (FIG. 8A).

0.5 ml of a 10 mg/ml sodium hyaluronate solution (FIG. 8B) and 0.5 ml of a 3% peptides ① water solution (FIG. 8C) were injected by the same method.

In can be seen from the result graph that in FIG. 8C, a cushion layer formed by injected peptides is fine and tight, and not loose. The sodium hyaluronate solution in FIG. 8B had diffused, whereas the normal saline in FIG. 8A did not form a sufficient support between a mucosa layer and a submucosal layer.

### Example 7. Experiments for Colonic Submucosal Injection of Rabbits

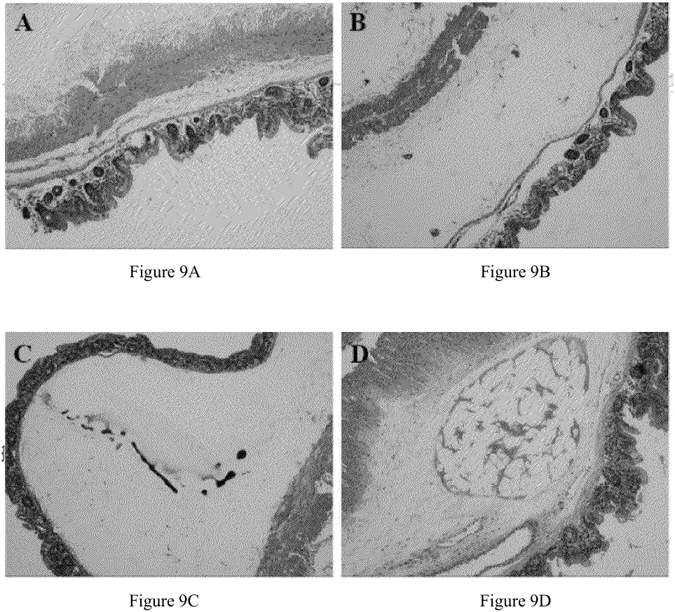

A New Zealand rabbit was fastened for 24 hours, injection of anesthesia was conducted via the ear vein, the abdomen was then opened, afterwards an open cutting was conducted along a midline of a colon to expose the colonic mucosa, 0.5 ml of normal saline was injected into the submucosal layer using a 25G needle. After observing for 30 min, the rabbit was sacrifice, a tissue was then taken out and fixed in formalin for 2 days, embedded by paraffin, sectioned, and finally stained with hematoxylin and eosin (FIG. 9B).

0.5 ml of a 10 mg/ml sodium hyaluronate solution (FIG. 9C) and 0.5 ml of a 3% peptide aqueous solution ① (FIG. 9D) were injected by the same method. An untreated normal tissue was used as a control (FIG. 9A).

It can be seen from the result graph that, in comparison with a normal tissue (FIG. 9A), in FIG. 9D it is obviously observed that under the fine and close lifted mucosa it was filled with the peptide gel, and that there was no loose sign after the experimental procedures of sectioning, fixing and staining e.g.. In FIG. 9C, sodium hyaluronate had flowed out, only partially filling achieved, and repeated injections were often required in the surgery to achieve the purpose of continuous filling. Whereas, in FIG. 9B, the normal saline also failed to complete the purpose of filling.

Finally, it should be noted that the above examples are merely intended to explain the technical solution of the present disclosure and not to limit the present disclosure; although the present disclosure was illustrated in detail with reference to the aforementioned examples, those of ordinary skill in the art should understand that they can still make modifications to the technical solution cited in the aforementioned examples, or make equivalent replacements to a portion of or all of the technical features; and these modifications or replacements do not make the essence of the corresponding technical solution depart from the scope of the technical solution of the examples of the present disclosure.

What is claimed is:

1. A self-assembling peptide, wherein, the self-assembling peptide sequence has a general formula of:
AC-Pro-X1-X3-X2-X3-X1-X3-X2-Pro-amide;
wherein, the N terminal is acetyl, and the C terminal is an amide group;
X1 is independently a positively charged amino acid, X2 is independently a negatively charged amino acid, and X3 is independently a hydrophobic amino acid.

2. The self-assembling peptide according to claim 1, wherein the X1s comprise one or more of Lys, Arg or His.

3. The self-assembling peptide according to claim 1, wherein the X2s comprise Asp and/or Glu.

4. The self-assembling peptide according to claim 1, wherein the X3s comprise one or more of Val, Leu, Ile or Phe.

5. A preparation method for the self-assembling peptide of claim 1, wherein the preparation method comprises a solid-phase peptide synthesis method.

6. A type of self-assembling peptide formulations, comprising the self-assembling peptide of claim 1.

7. The self-assembling peptide formulations according to claim 6, wherein the dosage form of the self-assembling peptide formulation comprises powder or a liquid preparation.

8. The self-assembling peptide formulations according to claim 6, further comprise pharmaceutically acceptable carriers and/or excipients.

9. A method for preparing (a) a hemostatic material; (b) a mucosal filler; or (c) an anti-adhesion agent, comprising of the self-assembling peptide of claim 1.

10. A peptide according to claim 1, wherein the self-assembling peptide consists of a sequence as shown in SEQ ID NO.1:
AC-Pro-Arg-Val-Asp-Val-Arg-Val-Asp-Pro-amide.

11. A peptide according to claim 1, wherein the self-assembling peptide consists of a sequence as shown in SEQ ID NO.2:
AC-Pro-Lys-Val-Glu-Val-Lys-Val-Glu-Pro-amide.

12. (canceled)

13. (canceled)

14. The self-assembling peptide formulation according to claim 6, wherein the self-assembling peptide is present at a concentration of 0.1%-99% in the preparation.

15. The self-assembling peptide formulation according to claim 6, wherein, the self-assembling peptide is present at a concentration of no greater than 4% in the preparation.

16. The self-assembling peptide formulation according to claim 6, wherein the self-assembling peptide is present at a concentration of 4%, 3%, 2% or 1% in the formulation.
